(19) United States
(12) Patent Application Publication
(10) Pub. No.: US 2009/0163971 A1
(43) Pub. Date: Jun. 25, 2009

(54) THERAPY CALCULATION AND THERAPY DELIVERY MODES

(75) Inventors: Yinghong Yu, Shoreview, MN (US); Jiang Ding, Shoreview, MN (US)

Correspondence Address: SCHWEGMAN, LUNDBERG & WOESSNER/BSC-CRM, PO BOX 2938, MINNEAPOLIS, MN, 55402, US

(73) Assignee: Cardiac Pacemakers, Inc., St. Paul, MN (US)

(21) Appl. No.: 12/336,036

(22) Filed: Dec. 16, 2008

Related U.S. Application Data
(60) Provisional application No. 61/008,342, filed on Dec. 19, 2007.

(51) Int. Cl. A61N 1/365

(52) U.S. Cl. 607/17

(57) ABSTRACT

A therapy can be provided using a therapy control parameter calculated using physiological data received at a first time. The therapy can be inhibited at a second time and the therapy control parameter can be recalculated using physiological data received at or near the second time while the therapy is inhibited, and the therapy can be provided using the recomputed therapy control parameter.

### CROSS-REFERENCE TO RELATED APPLICATIONS

This application claims the benefit under 35 U.S.C. 119(e) of U.S. Provisional Application No. 61/008,342, filed on Dec. 19, 2007, which is hereby incorporated by reference in its entirety.

### TECHNICAL FIELD

This document pertains generally to implantable medical devices, and more particularly, but not by way of limitation, to therapy calculation and therapy delivery modes.

### BACKGROUND

Cardiac rhythm or function management devices can include implantable devices to help maintain heart rhythm or function. Cardiac rhythm or function management devices can include pacers, defibrillators, cardioverters, cardiac resynchronization therapy (CRT), or various combinations of these or other devices. In various examples, cardiac rhythm or function management devices can sense intrinsic heart contractions, deliver pacing pulses to evoke responsive heart contractions, or deliver a shock to interrupt certain arrhythmias. In certain examples, one or more of these functions can help improve a patient's heart rhythm or can help coordinate a spatial nature of a heart contraction, either of which can improve cardiac output of blood to help meet the patient's metabolic need for such cardiac output.

### OVERVIEW

A therapy can be provided using a therapy control parameter calculated using physiological data received at a first time. The therapy can be inhibited at a second time and the therapy control parameter can be recalculated using physiological data received at or near the second time while the therapy is inhibited, and the therapy can be provided using the recomputed therapy control parameter.

In Example 1, a system includes an implantable medical device including at least one of an atrial sensing channel configured to receive an intrinsic atrial activation, a left ventricular sensing channel configured to receive an intrinsic left ventricular activation, or a right ventricular sensing channel configured to receive an intrinsic right ventricular activation. The system further includes a processor configured to receive physiological data from a subject at a first time and to calculate a therapy control parameter including at least one of an atrioventricular (AV) delay or a biventricular offset (BVO) using the physiological data received at the first time, the physiological data including at least one of an intrinsic atrioventricular interval AV_{R} determined using the received intrinsic atrial activation and the received intrinsic right ventricular activation, an intrinsic atrioventricular interval AV_{L} determined using the received intrinsic atrial activation and the received intrinsic left ventricular activation, or an intrinsic interventricular interval Δ_{RL} determined using the received intrinsic left ventricular activation and the received intrinsic right ventricular activation, wherein the implantable medical device is configured to provide a therapy to the subject using the computed therapy control parameter, wherein the processor is configured to inhibit the therapy at a second time, to receive the physiological data from the subject at or near the second time while the therapy is inhibited, and to recalculate the therapy control parameter using the physiological data received at or near the second time, and wherein the implantable medical device is configured to deliver the therapy to the subject using the recomputed therapy control parameter.

In Example 2, the processor of Example 1 is optionally configured to calculate the therapy control parameter including the AV delay using the physiological data including the AV_{R} and the AV_{L}.

In Example 3, the processor of any one or more of Examples 1-2 is optionally configured to calculate the therapy control parameter including the AV delay using the physiological data including the Δ_{RL}, wherein the Δ_{RL} includes the time between the right ventricular intrinsic activation and the left ventricular intrinsic activation.

In Example 4, the processor of any one or more of Examples 1-3 is optionally configured to calculate the therapy control parameter including the BVO using the physiological data including the AV_{R} and the AV_{L}.

In Example 5, the processor of any one or more of Examples 1-4 is optionally configured to calculate the therapy control parameter including the BVO using the physiological data including the Δ_{RL}, wherein the Δ_{RL} includes the time between the right ventricular intrinsic activation and the left ventricular intrinsic activation.

In Example 6, the implantable medical device of any one or more of Examples 1-5 is optionally configured to inhibit the therapy at the second time in response to a remote suspend therapy request received during the first therapy.

In Example 7, the implantable medical device of any one or more of Examples 1-6 is optionally configured to inhibit the therapy at the second time in response to a regularly scheduled suspend therapy request configured to provide a regular chance to recomputed the therapy control parameter.

In Example 8, the regularly scheduled suspend therapy request of any one or more of Examples 1-7 optionally includes after a predetermined time interval.

In Example 9, the regularly scheduled suspend therapy request of any one or more of Examples 1-8 optionally includes after a predetermined number of cardiac cycles.

In Example 10, the implantable medical device of any one or more of Examples 1-9 is optionally configured to compare the using the physiological data received at the first time to the physiological data received at or near the second time, and to provide an alert using the results of the comparison.

In Example 11, a method includes receiving physiological data from a subject at a first time, calculating a therapy control parameter including at least one of an atrioventricular (AV) delay or a biventricular offset (BVO) using the physiological data received at the first time, the physiological data including at least one of an intrinsic atrioventricular interval AV_{R} determined using a received intrinsic atrial activation and a received intrinsic right ventricular activation, an intrinsic atrioventricular interval AV_{L} determined using a received intrinsic atrial activation and a received intrinsic left ventricular activation, or an intrinsic interventricular interval Δ_{RL} determined using a received intrinsic left ventricular activation and a received intrinsic right ventricular activation, wherein the method further includes providing a therapy to the subject using the calculated therapy control parameter, inhibiting the therapy at a second time, receiving physiological data from the subject at or near the second time while the therapy is inhibited, recalculating the therapy control parameter using the physiological data received at or near the second time, and providing the therapy to the subject using the recomputed therapy control parameter.

In Example 12, the calculating the therapy control parameter of Example 11 optionally includes calculating the AV delay using the physiological data including the AV_{R} and the AV_{L}.

In Example 13, the calculating the therapy control parameter of any one or more of Examples 11-12 optionally includes calculating the AV delay using the physiological data including the Δ_{RL}, wherein the Δ_{RL} includes the time between the right ventricular intrinsic activation and the left ventricular intrinsic activation.

In Example 14, the calculating the therapy control parameter of any one or more of Examples 11-13 optionally includes calculating the BVO using the physiological data including the AV_{R} and the AV_{L}.

In Example 15, the calculating the therapy control parameter of any one or more of Examples 11-14 optionally includes calculating the BVO using the physiological data including the Δ_{RL}, wherein the Δ_{RL} includes the time between the right ventricular intrinsic activation and the left ventricular intrinsic activation.

In Example 16, the inhibiting the therapy at the second time of any one or more of Examples 11-15 optionally includes in response to a remote suspend therapy request received during the first therapy.

In Example 17, the inhibiting the therapy at the second time of any one or more of Examples 11-16 optionally includes inhibiting in response to a regularly scheduled suspend therapy request configured to provide a regular chance to recomputed the therapy control parameter.

In Example 18, the inhibiting in response to the regularly scheduled suspend therapy request of any one or more of Examples 11-17 optionally includes inhibiting after a predetermined time interval.

In Example 19, the inhibiting in response to the regularly scheduled suspend therapy request of any one or more of Examples 11-18 optionally includes inhibiting after a predetermined number of cardiac cycles.

In Example 20, the method of any one or more of Examples 11-19 optionally includes comparing the physiological data received at the first time to the physiological data received at or near the second time, and to provide an alert using the results of the comparison.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIGS. 1 and 2 illustrate generally examples of systems or portions of a system for delivering cardiac therapy.

FIG. 3 illustrates generally an example of a state diagram illustrating switching between a therapy deliver mode and a therapy calculation mode.

### DETAILED DESCRIPTION

Generally, heart failure (HF) refers to a cardiac disorder that impairs the ability of a heart to pump a sufficient amount of blood through a body. HF can be due to a variety of etiologies, e.g., ischemic heart disease. Certain HF subjects suffer from some degree of AV block such that their cardiac output can be improved by synchronizing atrial and ventricular contractions using dual chamber pacing having a specified atrioventricular (AV) delay interval. Dual chamber pacing can include pacing where energy is delivered to both at least one atrium and at least one ventricle. The AV delay interval, as used herein, refers to the interval between an atrial event (e.g., an atrial pace or an atrial sense, usually the right atrium) and a first ventricular pace to one of the ventricles (e.g., a right ventricle). The AV delay interval can be the same or different depending upon whether it is initiated by an atrial sense or an atrial pace (e.g., in atrial tracking mode or AV sequential pacing mode, respectively). Bi-ventricular pacing includes pacing both the left ventricle and the right ventricle. The biventricular offset (BVO) interval, as used herein, refers to the interval between the first ventricular pace and a second ventricular pace to the other ventricle (e.g., the left ventricle) during the same cardiac cycle. One approach to bi-ventricular pacing includes specifying an AV delay interval and a BVO interval. Another approach to bi-ventricular pacing includes specifying a separate AV delay interval for each ventricle, which can be designated as AV_{R} for the right ventricle and AV_{L} for the left ventricle. In subjects having normal AV conduction, the optimal or desired AV delay and BVO intervals can be related to both the intrinsic atrioventricular interval and the amount of pre-excitation time needed for one ventricle relative to the other (e.g., the extent of the ventricular conduction deficit).

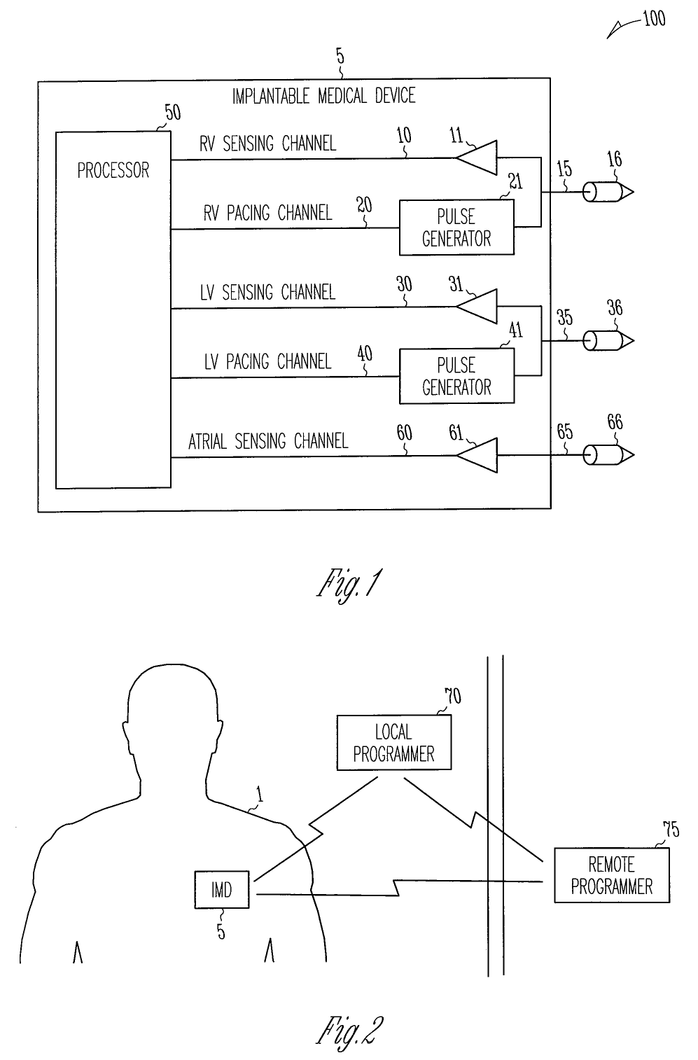

FIG. 1 illustrates generally an example of a system 100 for delivering cardiac therapy. In an example, the system 100 can include an implantable medical device (IMD) 5 having a processor 50, a right ventricular sensing channel 10, a right ventricular pacing channel 20, a left ventricular sensing channel 30, a left ventricular pacing channel 40, and an atrial sensing channel 60. The atrial sensing channel 60 can include at least one of a right atrial sensing channel or a left atrial sensing channel. In other examples, the IMD 5 can include a combination of at least one of the a right ventricular sensing channel 10, the right ventricular pacing channel 20, the left ventricular sensing channel 30, the left ventricular pacing channel 40, or the atrial sensing channel 60.

In certain examples, the right ventricular sensing channel 10 can include a sense amplifier 11, the left ventricular sensing channel 30 can include a sense amplifier 31, the right ventricle pacing channel 20 can include a pulse generator 21, the left ventricular pacing channel 40 can include a pulse generator 41, and the atrial sensing channel 60 can include a sense amplifier 61. In other examples, the right ventricular sensing channel 10 or the right ventricular pacing channel 20 can be coupled to an electrode 16 disposed on a lead 15 or elsewhere, the left ventricular sensing channel 30 or the left ventricular pacing channels 40 can be coupled to an electrode 36 disposed on a lead 35 or elsewhere, or the atrial sensing channel 60 can be coupled to an electrode 66 disposed on a lead 65 or elsewhere.

In certain examples, the lead 15 can be configured to electrically couple the sense amplifier 11 or the pulse generator 21 to the electrode 16, which can be configured to be located in a right ventricle, such as in the septal region, the free wall region, or another region of the right ventricle. Similarly, the lead 35 can be configured to electrically couple the sense amplifier 31 or the pulse generator 41 to the electrode 36, which can be configured to be located in, on, or near a left ventricle, such as in the septal region, the free wall region, or another region of the left ventricle or in the coronary vasculature. Further, the lead 65 can be configured to electrically couple the sense amplifier 61 to the electrode 66, which can be configured to be located in at least one of a right atrium or a left atrium of the subject 101.

In certain examples, the implantable medical device 5 can include one or more other pacing or sensing channels, such as an internal thoracic pacing or sensing channel configured to couple the processor 50 to an internal thoracic location external to the heart (e.g., through one or more leads, electrodes, pulse generators, or sense amplifiers). In an example, the internal thoracic pacing or sensing channel can be configured to send or receive information to or from a housing can electrode, located on the exterior housing of an implantable medical device located in the internal thoracic location external to the heart.

In the example of FIG. 1, the processor 50 can be an implantable component, an external component, or a combination or permutation of an implantable processor and an external processor. In an example, if at least a portion of the processor 50 includes an external processor, then the processor 50 can be configured to be communicatively coupled (such as via telemetry, RF, or other communication protocol) with the remaining implantable components (such as the sense amplifier 11, 31, the pulse generator 21, 41, the lead 15, 35, or the electrode 16, 36). In an example, the implantable processor can be configured to have reduced or minimal functionality or power consumption. In certain examples, it can be advantageous for the processor 50 to include an external processor for computing complex operations, such as to compute an AV delay interval. In other examples, the external processor can include an external device that can be either local or remote. In an example, the processor 50 can include a microcontroller, a microprocessor, a logic circuit, or other processor.

FIG. 2 illustrates generally an example of a portion of a system 200 including an IMD 5 configured to be implanted in a subject 101. The system 200 can include at least one of a local programmer 70 or a remote programmer 75. Both the local programmer 70 and the remote programmer 75 are external components. In an example, the local programmer 70 can include a hand-held programmer or other programmer capable of being positioned in communication proximity to the processor 50. The proximity range between the processor 50 and the local programmer 70 can vary depending upon the type of data communication and is bound by the physical constraints of the communication type. In an example, the remote programmer 75 can include any programmer configured to communicate with the IMD 5 either directly or indirectly (such as through another device, e.g., a router, the local programmer 70, etc.). In various examples, the remote programmer 75 can be configured to communicate with or store information from a plurality of implanted or external devices, and the remote programmer 75 can be configured to be located a long distance from the subject 1.

In an example, the local programmer 70 or the remote programmer 75 can be configured to send information to or receive information from the IMD 5. The information can include programming information, subject data, device data, or other instructions, alerts, or other information. Further, the local programmer 70 or the remote programmer 75 can be configured to communicate the sent or received information to a user or physician, such as by sending an alert via email of the status of the subject 1 or the system components.

### Therapy Calculation and Therapy Deliver Modes

The present inventor has recognized, among other things, that it can be advantageous to update or calculate at least one therapy parameter as the physiological condition of a subject changes (e.g., as the condition gets better or gets worse). As such, in an example, a therapy can be suspended (e.g., completely shut off) to allow for intrinsic or other physiological data (e.g., intrinsic cardiac intervals) to be measured in order to calculate, update, or otherwise modulate the at least one therapy parameter (e.g., an AV delay, a BVO, etc.) Therapy deliver mode, as used herein, refers to a mode of an implantable medical device where the device is delivering or capable of delivering therapy to a subject (e.g., a pacing therapy, etc.). In certain examples, the therapy delivery mode can include an ambulatory mode. Generally, ambulatory care is any medical care delivered on an outpatient basis, or generally, medical care outside of a hospital or outside of direct contact with a clinician. Once an implantable device (e.g., IMD 5) has been implanted, programmed, and is in use (e.g., delivering therapy), it can be considered to be in therapy delivery mode or ambulatory mode. Certain therapies, such as pacing therapy, can involve the detection or sensing of intrinsic intervals between cardiac events or contractions in order to program, calculate, or determine the appropriate therapy. One such example is computing an optimal or desirable AV delay, such as disclosed in the commonly assigned Ding et al. U.S. Pat. No. 7,013,176 entitled, “METHOD AND APPARATUS FOR SETTING PACING PARAMETERS IN CARDIAC RESYNCHRONIZATION THERAPY,” (herein “the Ding et al. '176 patent”) the commonly assigned Ding et al. U.S. Pat. No. 7,203,540 entitled, “METHOD AND SYSTEM FOR SETTING CARDIAC RESYNCHRONIZATION THERAPY PARAMETERS,” (herein “the Ding et al. '540 patent”) or the commonly assigned Ding et al. U.S. Pat. No. 7,123,960 entitled, “METHOD AND SYSTEM FOR DELIVERING CARDIAC RESYNCHRONIZATION THERAPY WITH VARIABLE ATRIO-VENTRICULAR DELAY,” (herein “the Ding et al. '960 patent”) the disclosures of which are each incorporated by reference in their entirety, including their disclosure of computing an optimal or desirable AV delay interval. Another example includes computing an AV delay or a BVO, such as disclosed in the commonly assigned Yu et al. U.S. Provisional Patent Application entitled, “DETERMINATION OF STIMULATION DELAY BETWEEN VENTRICULAR SITES,” filed on Oct. 29, 2007 (Attorney Docket No. 279.B73PRV), the disclosure of which is incorporated by reference in its entirety, including its disclosure of computing a BVO. In other examples, other methods of calculating or computing an AV delay interval, a BVO, or some other pacing parameter can be used herein.

In an example, in order to calculate the appropriate AV delay interval (or the BVO interval), the device (e.g., IMD 5) can detect various intrinsic intervals (e.g., AV_{R}, the AV_{L}, the Δ_{RL} (wherein Δ_{RL} can include the difference between the AV_{R} and the AV_{L}, which, in certain examples, can be correlated to the width of the QRS wave), etc.) After the device has calculated the AV delay interval (or the BVO interval), the intrinsic intervals can be supplanted by the delivered cardiac therapy. Once the device is delivering therapy, or after the device has calculated its given therapy parameters and is monitoring the subject and ready to give therapy, the device can be said to be in therapy delivery mode. When the device is detecting (e.g., initially detecting, detecting, or updating) physiological data (such as, for example, the various intrinsic intervals) in order to determine, calculate, or recalculate a therapy control parameter (such as the appropriate AV delay interval) it can be said to be in therapy calculation mode.

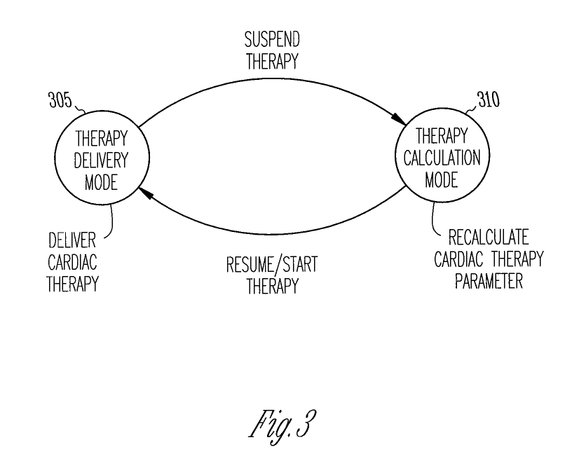

FIG. 3 illustrates generally an example of a state 300 diagram illustrating switching between a therapy delivery mode 305 and a therapy calculation mode 310.

During the therapy delivery mode 305, a therapy (e.g., a pacing or other therapy) is being delivered, e.g., as needed, using an implantable device. In an example, the implantable device can include a pacer and the therapy can include a pacing therapy having as a therapy control parameter at least one of an AV delay or a BVO. In other examples, the implantable device can include any such device configured to provide ambulatory therapy.

In an example, if the implantable device receives a suspend therapy request during the therapy delivery mode 305, then the therapy calculation mode 310 is entered. During the therapy calculation mode 310, therapy can be temporarily stopped or inhibited in order to allow the implantable device to calculate or recalculate at least one therapy control parameter. In an example, once the at least one therapy control parameter has been calculated or recalculated, then the therapy delivery mode can be entered and the therapy can be started or resumed.

In an example, the suspend therapy request can include a programmed or scheduled suspend therapy request put in place to automatically regularly update the at least one therapy parameter using intrinsic or unassisted physiological information from the subject. In other examples, the suspend therapy request can come from the subject or a clinician using the local programmer 70, or from a remote user or clinician using the remote programmer 75. In certain examples, the remote user or clinician can include a user or clinician located in a different country, state, city, building, or even room as the subject. In an example, the remote suspend therapy request can be received from the remote programmer 75 by the local programmer 70, and then transferred to the implantable device.

In an example, during the therapy delivery mode at 305, pacing can be delivered to a subject using an implantable pacer having a calculated desired or optimal AV delay interval. After a specified period of time, or upon receiving a suspend therapy request from the subject, a clinician, or other user, the implantable pacer can enter the therapy calculation mode at 310. In therapy calculation mode, pacing is suspended and intrinsic control of the heart returns. Here, the desired or optimal AV delay can be recalculated using measured intrinsic intervals (e.g., AV_{R}, AV_{L}, etc.) Once the desired or optimal AV delay has been recalculated, the implantable pacer can enter therapy delivery mode at 305, and therapy resumes (e.g., automatically resumes) with the newly calculated or updated desired or optimal AV delay.

In certain examples, if the intrinsic condition of the subject changes (e.g., changes unexpectedly), or if the intrinsic condition of the subject does not change (e.g., does not change when it is expected to change), then the detected information, or an alert, can be sent to a clinician or other user, such as by using at least one of the local programmer 70 or the remote programmer 75. In an example, using the remote programmer can include communicating to the local programmer 70 over a network or other long range communication medium.

### Additional Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown and described. However, the present inventor also contemplates examples in which only those elements shown and described are provided.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B.” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. A system comprising:
an implantable medical device including at least one of:
an atrial sensing channel configured to receive an intrinsic atrial activation;
a left ventricular sensing channel configured to receive an intrinsic left ventricular activation; or
a right ventricular sensing channel configured to receive an intrinsic right ventricular activation;
a processor configured to receive physiological data from a subject at a first time and to calculate a therapy control parameter including at least one of an atrioventricular (AV) delay or a biventricular offset (BVO) using the physiological data received at the first time, the physiological data including at least one of:
an intrinsic atrioventricular interval AV_{R} determined using the received intrinsic atrial activation and the received intrinsic right ventricular activation;
an intrinsic atrioventricular interval AV_{L} determined using the received intrinsic atrial activation and the received intrinsic left ventricular activation; or
an intrinsic interventricular interval Δ_{RL} determined using the received intrinsic left ventricular activation and the received intrinsic right ventricular activation;
wherein the implantable medical device is configured to provide a therapy to the subject using the computed therapy control parameter;
wherein the processor is configured to inhibit the therapy at a second time, to receive the physiological data from the subject at or near the second time while the therapy is inhibited, and to recalculate the therapy control parameter using the physiological data received at or near the second time; and
wherein the implantable medical device is configured to deliver the therapy to the subject using the recomputed therapy control parameter.

2. The system of claim 1, wherein the processor is configured to calculate the therapy control parameter including the AV delay using the physiological data including the AV_{R} and the AV_{L}.

3. The system of claim 1, wherein the processor is configured to calculate the therapy control parameter including the AV delay using the physiological data including the Δ_{RL}, wherein the Δ_{RL} includes the time between the right ventricular intrinsic activation and the left ventricular intrinsic activation.

4. The system of claim 1, wherein the processor is configured to calculate the therapy control parameter including the BVO using the physiological data including the AV_{R} and the AV_{L}.

5. The system of claim 1, wherein the processor is configured to calculate the therapy control parameter including the BVO using the physiological data including the Δ_{RL}, wherein the Δ_{RL} includes the time between the right ventricular intrinsic activation and the left ventricular intrinsic activation.

6. The system of claim 1, wherein the implantable medical device is configured to inhibit the therapy at the second time in response to a remote suspend therapy request received during the first therapy.

7. The system of claim 1, wherein the implantable medical device is configured to inhibit the therapy at the second time in response to a regularly scheduled suspend therapy request configured to provide a regular chance to recomputed the therapy control parameter.

8. The system of claim 7, wherein the regularly scheduled suspend therapy request includes after a predetermined time interval.

9. The system of claim 7, wherein the regularly scheduled suspend therapy request includes after a predetermined number of cardiac cycles.

10. The system of claim 1, wherein the implantable medical device is configured to compare the using the physiological data received at the first time to the physiological data received at or near the second time, and to provide an alert using the results of the comparison.

11. A method comprising:
receiving physiological data from a subject at a first time;
calculating a therapy control parameter including at least one of an atrioventricular (AV) delay or a biventricular offset (BVO) using the physiological data received at the first time, the physiological data including at least one of:
an intrinsic atrioventricular interval AV_{R} determined using a received intrinsic atrial activation and a received intrinsic right ventricular activation;
an intrinsic atrioventricular interval AV_{L} determined using a received intrinsic atrial activation and a received intrinsic left ventricular activation; or
an intrinsic interventricular interval Δ_{RL} determined using a received intrinsic left ventricular activation and a received intrinsic right ventricular activation;
providing a therapy to the subject using the calculated therapy control parameter;
inhibiting the therapy at a second time;
receiving physiological data from the subject at or near the second time while the therapy is inhibited;
recalculating the therapy control parameter using the physiological data received at or near the second time; and
providing the therapy to the subject using the recomputed therapy control parameter.

12. The method of claim 11, wherein the calculating the therapy control parameter includes calculating the AV delay using the physiological data including the AV_{R} and the AV_{L}.

13. The method of claim 11, wherein the calculating the therapy control parameter includes calculating the AV delay using the physiological data including the Δ_{RL}, wherein the Δ_{RL} includes the time between the right ventricular intrinsic activation and the left ventricular intrinsic activation.

14. The method of claim 11, wherein the calculating the therapy control parameter includes calculating the BVO using the physiological data including the AV_{R} and the AV_{L}.

15. The method of claim 11, wherein the calculating the therapy control parameter includes calculating the BVO using the physiological data including the Δ_{RL}, wherein the Δ_{RL} includes the time between the right ventricular intrinsic activation and the left ventricular intrinsic activation.

16. The method of claim 11, wherein the inhibiting the therapy at the second time includes in response to a remote suspend therapy request received during the first therapy.

17. The method of claim 11, wherein the inhibiting the therapy at the second time includes inhibiting in response to a regularly scheduled suspend therapy request configured to provide a regular chance to recomputed the therapy control parameter.

18. The method of claim 17, wherein the inhibiting in response to the regularly scheduled suspend therapy request includes inhibiting after a predetermined time interval.

19. The method of claim 17, wherein the inhibiting in response to the regularly scheduled suspend therapy request includes inhibiting after a predetermined number of cardiac cycles.

20. The method of claim 11, including comparing the physiological data received at the first time to the physiological data received at or near the second time, and to provide an alert using the results of the comparison.
